(19) United States
(12) Patent Application Publication
(10) Pub. No.: US 2025/0375406 A1
(43) Pub. Date: Dec. 11, 2025

(54) TREATMENT OF NON-ALCOHOLIC STEATOHEPATITIS AND NON-ALCOHOLIC FATTY LIVER DISEASE

(71) Applicant: VEROSCIENCE LLC, Tiverton, RI (US)

(72) Inventor: Anthony H. Cincotta, Tiverton, RI (US)

(21) Appl. No.: 18/874,384

(22) PCT Filed: Jun. 26, 2023
(86) PCT No.: PCT/US2023/026236

§ 371 Date: Dec. 12, 2024

Related U.S. Application Data
(60) Provisional application No. 63/355,432, filed on Jun. 24, 2022.

(51) Int. Cl. A61K 31/198; A61K 31/12; A61K 35/741; A61K 36/48; A61K 36/9066; A61P 1/16

(52) U.S. Cl. CPC A61K 31/198; A61K 31/12; A61K 35/741; A61K 36/48; A61K 36/9066; A61P 1/16

(57) ABSTRACT

Methods and compositions comprising one or more dopamine neuronal activity enhancers (e.g., dopamine receptor agonists), pantethine and solubilized curcumin for use in treating NAFLD and NASH are provided.

Methods and compositions comprising one or more dopamine neuronal activity enhancers (e.g., dopamine receptor agonists), pantethine and solubilized curcumin for use in treating NAFLD and NASH are provided.

### CROSS-REFERENCE TO RELATED APPLICATION

The present application claims priority to U.S. Provisional Application Ser. No. 63/355,432, filed on Jun. 24, 2022, the contents of which are hereby incorporated by reference in their entirety.

### TECHNICAL FIELD

This disclosure relates to methods for using compositions comprising one or more dopamine neuronal activity enhancers (e.g., dopamine receptor agonists) with gastrointestinal pro-health agents including for example anti-inflammatory agents such as prebiotics, antibiotics, or probiotics for the treatment of non-alcoholic steatohepatitis (NASH) in a subject in need thereof. The disclosure also relates to the treatment of non-alcoholic fatty liver disease (NAFLD).

### BACKGROUND

Non-alcoholic steatohepatitis (NASH) is a liver disease distinct from non-alcoholic fatty liver disease (NAFLD or hepatic steatosis or fatty liver). About 30-40% of the U.S. adult population has NAFLD, and about 8% have NASH. While NAFLD is characterized by simple increases in liver fat content (steatosis), NASH represents a significantly more severe and different liver disorder involving additional complex biochemical pathologies not observed in NAFLD. NAFLD patients do not typically develop complications from their liver disease. In contrast, NASH patients frequently develop life-threatening complications including, e.g., liver failure, cirrhosis, and liver cancer. NAFLD can be treated simply by reducing the lipid content of the liver. However, distinct from and unlike NAFLD, NASH represents a disease state where damage to liver tissue has occurred, thus requiring treatment that is not merely directed to reducing liver fat content but, more specifically and importantly, to repairing or preventing the progression of damage to liver tissue (e.g., lobular ballooning, fibrosis, inflammation, necrosis, apoptosis and oncogenesis) resulting from several co-existent pathological biochemical processes.

Unlike NAFLD, NASH is uniquely characterized by excessive and distinctive cellular and tissue steatosis, inflammation, alteration of macrophage/Kupffer cell polarization towards proinflammation, hepatocyte degeneration and cellular death, and fibrosis. NASH represents a disease state and pathological biochemistry wholly distinct from NAFLD. Pathologically, NASH is typically identifiable histologically by a composite of ballooning degeneration of hepatocytes, pericellular fibrosis, hepatic inflammation, necrosis, apoptosis, and micro vesicular and macro vesicular steatosis. NASH is also characterized by hepatic and adipose immunological imbalance and dysfunction that not only precipitates liver damage but can also contribute to hepatic vascular damage. NASH can typically be identified by liver biopsy, MRI scan, CT scan, or measurement of stable isotope incorporation into liver extracellular protein matrix (fibrosis indicator) and can further be assessed by quantifying levels of one or more biomarkers in the blood, e.g., plasma ALT, AST.

NAFLD, but not NASH, may respond quite favorably (i.e., remission of disease) to diet and exercise without any pharmacological intervention. There are also a few U.S. Food and Drug Administration (FDA) approved drugs for other diseases that have shown some promise in the treatment of NAFLD (e.g., pioglitazone, GLP-1 receptor agonists such as liraglutide, and sodium glucose cotransporter 2 (SGLT2) inhibitors). Importantly, therapies for several related metabolic disorders such as type 2 diabetes, dyslipidemia, and even NAFLD have proven ineffective in the treatment of NASH. Several drugs that have shown promise in the treatment of NAFLD have generally been either ineffective or not optimal in treating NASH, and exhibit untoward side effects (such as pioglitazone) limiting their potential use as therapies for NASH. That is, a preponderance of evidence clearly indicates that it is not a foregone conclusion that a therapy effective in treating type 2 diabetes, obesity, dyslipidemia, or NAFLD will always be safe and effective in treating NASH. This disease (NASH) is a disorder unto itself. Thus, there has been a long-recognized but unmet need for a practical, safe, and effective means of treating NASH.

### SUMMARY

The present disclosure relates generally to methods for the treatment of NAFLD or NASH including metabolic disorders associated with these conditions such as obesity, type 2 diabetes, prediabetes, hypertension, dyslipidemia, and renal disease, and cardiovascular disease (CVD) (e.g., coronary artery disease (including myocardial infarction), cerebrovascular disease (including stroke), and peripheral vascular disease), by modulating the activity of the CNS biological clock circuit with or without co-modulation of the inflammatory and neuro-modulatory state of the gastrointestinal (GI) tract.

The present disclosure also relates to simultaneously treating NAFLD or NASH along with obesity or other key metabolic disorders, including obesity, type 2 diabetes, prediabetes, hypertension, CVD, renal disease, and dyslipidemia.

Also disclosed herein are pharmaceutical formulations containing agents that increase central dopaminergic neuronal activity, such as bromocriptine or a pharmaceutically acceptable salt of bromocriptine, and pantethine with or without solubilized curcumin.

Further disclosed herein are pharmaceutical formulations containing agents that increase central dopaminergic neuronal activity, such as L-DOPA, a pharmaceutically acceptable salt of L-DOPA, or a biological source containing L-DOPA (e.g., broad bean or velvet bean (Mucuna pruriens) or extracts thereof (as is commercially available (e.g., from NutraCargo LLC or Ecuadorian Rainforest LLC) or produced by plant or seed extraction with an aqueous or organic solvent by commonly accepted chemical methods); and pantethine with or without solubilized curcumin.

Further disclosed herein is a method of treating non-alcoholic steatohepatitis (NASH) or non-alcoholic fatty liver disease (NAFLD) in a patient which comprises (i) administering to a patient in need of such treatment a pharmaceutical formulation containing agents that increase central dopaminergic neuronal activity, such as bromocriptine or a pharmaceutically acceptable salt of bromocriptine; and (ii) co-administering to the patient in need of such treatment pantethine with or without solubilized curcumin.

Further disclosed herein is a method of treating non-alcoholic steatohepatitis (NASH) or non-alcoholic fatty liver disease (NAFLD) in a patient which comprises (i) administering to a patient in need of such treatment L-DOPA or a pharmaceutically acceptable salt of L-DOPA, or a biological source containing L-DOPA (e.g., broad bean or velvet bean (Mucuna pruriens), oceanic biological sources or extracts thereof) and; (ii) co-administering to the patient in need of treatment pantethine with or without solubilized curcumin.

Further disclosed herein is a method of treating non-alcoholic steatohepatitis (NASH) or non-alcoholic fatty liver disease (NAFLD) in a patient which comprises (i) administering at a predetermined time of day to a patient in need of such treatment, a pharmaceutical formulation containing agents that increase central dopaminergic neuronal activity, such as bromocriptine or a pharmaceutically acceptable salt of bromocriptine and (ii) co-administering to the patient in need of treatment pantethine with or without solubilized curcumin.

Further disclosed herein is a method of treating non-alcoholic steatohepatitis (NASH) or non-alcoholic fatty liver disease (NAFLD) in a patient which comprises (i) administering at a predetermined time of day to a patient in need of such treatment L-DOPA or a pharmaceutically acceptable salt of L-DOPA, or a biological source containing L-DOPA (e.g., broad bean or velvet bean (Mucuna pruriens) or extracts thereof) and (ii) co-administering to the patient in need of treatment pantethine with or without solubilized curcumin.

Further disclosed herein is a method of treating non-alcoholic steatohepatitis (NASH) or non-alcoholic fatty liver disease (NAFLD) in a patient which comprises (i) administering within 4 hours of waking from the daily sleep cycle (e.g., in the morning in humans) to a patient in need of such treatment pharmaceutical formulations containing agents that increase central dopaminergic neuronal activity such as bromocriptine or a pharmaceutically acceptable salt of bromocriptine and (ii) co-administering to the patient in need of treatment pantethine with or without solubilized curcumin.

Further disclosed herein is a method of treating non-alcoholic steatohepatitis (NASH) or non-alcoholic fatty liver disease (NAFLD) in a patient which comprises (i) administering to a patient in need of such treatment between 0400 and 1200 hours a pharmaceutical formulation containing agents that increase central dopaminergic neuronal activity such as bromocriptine or a pharmaceutically acceptable salt of bromocriptine and (ii) co-administering between 0400 and 1200 hours to the patient in need of treatment pantethine with or without solubilized curcumin.

Further disclosed herein is a method of treating non-alcoholic steatohepatitis (NASH) or non-alcoholic fatty liver disease (NAFLD) in a patient which comprises (i) administering within 4 hours of waking from the daily sleep cycle to a patient in need of such treatment L-DOPA, or a pharmaceutically acceptable salt of L-DOPA, or a biological source containing L-DOPA (e.g., broad bean or velvet bean (Mucuna pruriens) or extracts thereof) and (ii) co-administering to the patient in need of treatment pantethine with or without solubilized curcumin.

Further disclosed herein is a method of treating non-alcoholic steatohepatitis (NASH) or non-alcoholic fatty liver disease (NAFLD) in a patient which comprises administering to a patient in need of such treatment between 0400 and 1200 hours (i) L-DOPA or a pharmaceutically acceptable salt of L-DOPA, or a biological source containing L-DOPA (e.g., broad bean or velvet bean (Mucuna pruriens) or extracts thereof, and (ii) co-administering between 0400 and 1200 hours pantethine with or without solubilized curcumin to the patient in need of such treatment and optionally administering a probiotic to the patient in need of such treatment.

Further disclosed herein is a method of treating non-alcoholic steatohepatitis (NASH) or non-alcoholic fatty liver disease (NAFLD) in a patient which comprises administering to a patient in need of such treatment (i) a pharmaceutical formulation containing agents that increase central dopaminergic neuronal activity such as bromocriptine citrate, within 4 hours of waking from the daily sleep cycle, and, (ii) co-administering to the patient in need of such treatment pantethine with or without solubilized curcumin.

Further disclosed herein is a method of treating non-alcoholic steatohepatitis (NASH) or non-alcoholic fatty liver disease (NAFLD) in a patient which comprises administering to a patient in need of such treatment (i) a pharmaceutical formulation containing agents that increase central dopaminergic neuronal activity, such as bromocriptine citrate within 4 hours of waking from the daily sleep cycle, and (ii) co-administering to the patient in need of such treatment within 4 hours of waking from the daily sleep cycle pantethine with or without solubilized curcumin.

Further disclosed herein is a method of treating non-alcoholic steatohepatitis (NASH) or non-alcoholic fatty liver disease (NAFLD) in a patient which comprises administering to a patient in need of such treatment (i) bromocriptine citrate within 4 hours of waking from the daily sleep cycle; (ii) co-administering to the patient in need of such treatment within 4 hours of waking pantethine with or without solubilized curcumin; and (iii) administering a probiotic to the patient in need of such treatment.

Further disclosed herein is a method of treating non-alcoholic steatohepatitis (NASH) or non-alcoholic fatty liver disease (NAFLD) in a patient which comprises administering to a patient in need of such treatment (i) L-DOPA or a pharmaceutically acceptable salt of L-DOPA, or a biological source containing L-DOPA (e.g., broad bean or velvet bean (Mucuna pruriens) or extracts thereof) within 4 hours of waking, and (ii) co-administering to the patient in need of such treatment pantethine, solubilized curcumin and a probiotic within 4 hours of waking from the daily sleep cycle.

Further disclosed herein is a method of treating non-alcoholic steatohepatitis (NASH) or non-alcoholic fatty liver disease (NAFLD) in a patient which comprises administering to a patient in need of such treatment (i) L-DOPA or a pharmaceutically acceptable salt of L-DOPA, or a biological source containing L-DOPA (e.g., broad bean or velvet bean (Mucuna pruriens) or extracts thereof within 4 hours of waking from the daily sleep cycle, and (ii) optionally co-administering to the patient in need of such treatment at least one of pantethine, solubilized curcumin and a probiotic within 4 hours of waking from the daily sleep cycle.

Further disclosed herein is a method of treating non-alcoholic steatohepatitis (NASH) or non-alcoholic fatty liver disease (NAFLD) in a patient which comprises administering to a patient in need of such treatment (i) pharmaceutical formulations containing agents that increase central dopaminergic neuronal activity, such as bromocriptine citrate, within 2 hours of waking from the daily sleep cycle; (ii) co-administering to the patient in need of such treatment pantethine with or without solubilized curcumin within 2 hours of waking from the daily sleep cycle; and (iii) administering a probiotic to the patient in need of such treatment.

Further disclosed herein is a method of treating non-alcoholic steatohepatitis (NASH) or non-alcoholic fatty liver disease (NAFLD) in a patient which comprises administering to a patient in need of such treatment (i) L-DOPA or a pharmaceutically acceptable salt of L-DOPA, or a biological source containing L-DOPA (e.g., broad bean or velvet bean (Mucuna pruriens) or extracts thereof within 2 hours of waking from the daily sleep cycle; (ii) co-administering to the patient in need of such treatment pantethine and solubilized curcumin within 2 hours of waking from the daily sleep cycle; and (iii) administering a probiotic to the patient in need of such treatment.

Further disclosed herein is a method of simultaneously treating non-alcoholic steatohepatitis (NASH) or non-alcoholic fatty liver disease (NAFLD) and obesity in a patient which comprises administering to a patient in need of such treatment (i) a pharmaceutical formulation containing agents that increase central dopaminergic neuronal activity such as bromocriptine citrate, within 4 hours of waking from the daily sleep cycle; (ii) co-administering pantethine with or without solubilized curcumin to the patient in need of such treatment; and (iii) administering a probiotic to the patient in need of such treatment.

Further disclosed herein is a method of simultaneously treating non-alcoholic steatohepatitis (NASH) or non-alcoholic fatty liver disease (NAFLD) and obesity in a patient which comprises administering to a patient in need of such treatment (i) L-DOPA or a pharmaceutically acceptable salt of L-DOPA, or a biological source containing L-DOPA (e.g., broad bean or velvet bean (Mucuna pruriens) or extracts thereof, within 4 hours of waking from the daily sleep cycle; (ii) co-administering pantethine and solubilized curcumin to the patient in need of such treatment; and (iii) administering a probiotic to the patient in need of such treatment.

Further disclosed herein is a method of simultaneously treating non-alcoholic steatohepatitis (NASH) or non-alcoholic fatty liver disease (NAFLD) and obesity in a patient which comprises administering to a patient in need of such treatment (i) bromocriptine citrate within 2 hours of waking from the daily sleep cycle; (ii) co-administering pantethine and solubilized curcumin within 2 hours of waking from the daily sleep cycle; and (iii) administering a probiotic to the patient in need of such treatment.

Further disclosed herein is a method of simultaneously treating non-alcoholic steatohepatitis (NASH) or non-alcoholic fatty liver disease (NAFLD) and obesity in a patient which comprises administering to a patient in need of such treatment (i) L-DOPA or a pharmaceutically acceptable salt of L-DOPA, or a biological source containing L-DOPA (e.g., broad bean or velvet bean (Mucuna pruriens) or extracts thereof within 2 hours of waking; (ii) co-administering pantethine with or without solubilized curcumin within 2 hours of waking; and (iii) administering a probiotic to the patient in need of such treatment.

Further disclosed herein is a method of treating non-alcoholic steatohepatitis (NASH) or non-alcoholic fatty liver disease (NAFLD) and obesity or metabolic disorders (prediabetes, glucose intolerance, hypertension, dyslipidemia, type 2 diabetes, cardiovascular disease) in a patient which comprises administering to a patient in need of such treatment within 4 hours of waking from the daily sleep cycle (i) L-DOPA or a pharmaceutically acceptable salt of L-DOPA, or a biological source containing L-DOPA (e.g., broad bean or velvet bean (Mucuna pruriens) or extracts thereof; (ii) co-administering pantethine with or without solubilized curcumin to the patient in need of such treatment; and (iii) optionally administering a probiotic to the patient in need of such treatment.

Further disclosed herein is a method of treating non-alcoholic steatohepatitis (NASH) or non-alcoholic fatty liver disease (NAFLD) and metabolic disorders (obesity, prediabetes, glucose intolerance, hypertension, dyslipidemia, type 2 diabetes, cardiovascular disease) in a patient which comprises administering to a patient in need of such treatment between 0400 and 1200 hours (i) L-DOPA or a pharmaceutically acceptable salt of L-DOPA, or a biological source containing L-DOPA (e.g., broad bean or velvet bean (Mucuna pruriens) or extracts thereof; (ii) co-administering between 0400 and 1200 hours pantethine with or without solubilized curcumin to the patient in need of such treatment; and (iii) optionally administering a probiotic to the patient in need of such treatment.

Further disclosed herein is a method of treating non-alcoholic steatohepatitis (NASH) or non-alcoholic fatty liver disease (NAFLD) and obesity or metabolic disorders (prediabetes, glucose intolerance, hypertension, dyslipidemia, type 2 diabetes, cardiovascular disease) in a patient which comprises administering to a patient in need of such treatment within 4 hours of waking from the daily sleep cycle (i) L-DOPA or a pharmaceutically acceptable salt of L-DOPA, or a biological source containing L-DOPA (e.g., broad bean or velvet bean (Mucuna pruriens) or extracts thereof; (ii) co-administering within 4 hours of waking from the daily sleep cycle pantethine with or without solubilized curcumin to the patient in need of such treatment; and (iii) optionally administering a probiotic to the patient in need of such treatment.

Further disclosed herein is a method of treating non-alcoholic steatohepatitis (NASH) or non-alcoholic fatty liver disease (NAFLD) in a patient which comprises administering to a patient in need of such treatment within 4 hours of waking from the daily sleep cycle, (i) pantethine; (ii) solubilized curcumin; and (iii) optionally administering a probiotic.

Further disclosed herein is a method of treating non-alcoholic steatohepatitis (NASH) or non-alcoholic fatty liver disease (NAFLD, fatty liver) and obesity or metabolic disorders (prediabetes, glucose intolerance, hypertension, dyslipidemia, type 2 diabetes, cardiovascular disease) in a patient which comprises administering to a patient in need of such within 4 hours of waking from the daily sleep cycle (i) pantethine; (ii) solubilized curcumin; and (iii) optionally a probiotic.

Further disclosed herein is a method of treating non-alcoholic steatohepatitis (NASH) or non-alcoholic fatty liver disease (NAFLD) and obesity or metabolic disorders (prediabetes, glucose intolerance, hypertension, dyslipidemia, type 2 diabetes, cardiovascular disease) in a patient which comprises administering to a patient in need of such treatment within 4 hours of waking from the daily sleep cycle (i) L-DOPA or a pharmaceutically acceptable salt of L-DOPA, or a biological source containing L-DOPA (e.g., broad bean or velvet bean (Mucuna pruriens) or extracts thereof; (ii) pantethine; and (iii) solubilized curcumin.

Further disclosed herein is a method of treating non-alcoholic steatohepatitis (NASH) or non-alcoholic fatty liver disease (NAFLD) and obesity or metabolic disorders (prediabetes, glucose intolerance, hypertension, dyslipidemia, type 2 diabetes, cardiovascular disease) in a patient which comprises administering to a patient in need of such treatment (i) L-DOPA or a pharmaceutically acceptable salt of L-DOPA, or a biological source containing L-DOPA (e.g., broad bean or velvet bean (Mucuna pruriens) or extracts thereof; (ii) pantethine; and (iii) solubilized curcumin, in a manner that effectuates peak plasma concentrations of L-DOPA, pantethine, and curcumin at a time period of the day that is within the circadian peak of central dopaminergic activity in a healthy individual.

Further disclosed herein is a method of treating non-alcoholic steatohepatitis (NASH) or non-alcoholic fatty liver disease (NAFLD) and obesity or metabolic disorders (prediabetes, glucose intolerance, hypertension, dyslipidemia, type 2 diabetes, cardiovascular disease) in a patient which comprises administering to a patient in need of such treatment (i) L-DOPA or a pharmaceutically acceptable salt of L-DOPA, or a biological source containing L-DOPA (e.g., broad bean or velvet bean (Mucuna pruriens) or extracts thereof; (ii) pantethine; and (iii) solubilized curcumin, in a manner that increases central dopaminergic neuronal activity within the time of day of the circadian peak of central dopaminergic activity in a healthy individual.

Further disclosed herein is a method of increasing the circadian peak of central dopaminergic neuronal activity in a patient with NAFLD or NASH which comprises administering to a patient in need of such treatment (i) L-DOPA or a pharmaceutically acceptable salt of L-DOPA, or a biological source containing L-DOPA (e.g., broad bean or velvet bean (Mucuna pruriens) or extracts thereof and (ii) pantethine, with or without solubilized curcumin, in a manner that increases central dopaminergic neuronal activity within the time of day of the circadian peak of central dopaminergic activity in a healthy individual of the same species and sex.

Further disclosed herein is a method of increasing the circadian peak of central dopaminergic neuronal activity in a patient with NAFLD or NASH which comprises administering to a patient in need of such treatment (i) L-DOPA or a pharmaceutically acceptable salt of L-DOPA, or a biological source containing L-DOPA (e.g., broad bean or velvet bean (Mucuna pruriens) or extracts thereof and (ii) pantethine, with or without solubilized curcumin, in a manner that increases central dopaminergic neuronal activity within the time of day of the circadian peak of central dopaminergic activity in a healthy individual of the same species and sex, wherein such administration also treats NAFLD or NASH.

Further disclosed herein is a method of treating non-alcoholic steatohepatitis (NASH) in a patient which comprises administering to a patient in need of such treatment (i) L-DOPA or a pharmaceutically acceptable salt of L-DOPA, or a biological source containing L-DOPA (e.g., broad bean or velvet bean (Mucuna pruriens), or extracts thereof; (ii) pantethine; and (iii) optionally solubilized curcumin, so as to effectuate a peak in central dopaminergic neuronal activity that mimics that of a healthy individual of the same species and sex.

Further disclosed herein is a method of increasing the circadian peak of central dopaminergic neuronal activity in a patient which comprises administering to a patient in need of such treatment between 0400 and 1200 hours (i) L-DOPA or a pharmaceutically acceptable salt of L-DOPA, or a biological source containing L-DOPA (e.g., broad bean or velvet bean (Mucuna pruriens) or extracts thereof and (ii) pantethine, with or without solubilized curcumin.

Further disclosed herein is a method of increasing the circadian peak of central dopaminergic neuronal activity in a patient which comprises administering to a patient in need of such treatment between 0400 and 1200 hours (i) an agent that increases central dopaminergic neuronal activity such as bromocriptine or a pharmaceutically acceptable salt of bromocriptine and (ii) pantethine, with or without solubilized curcumin.

Further disclosed herein is a method of increasing the circadian peak of central dopaminergic neuronal activity in a patient which comprises administering to a patient in need of such treatment between 0400 and 1200 hours (i) pantethine and (ii) solubilized curcumin.

In some examples the curcumin and pantethine are administered simultaneously with the dopaminergic neuronal activity enhancer. In other embodiments, the curcumin and pantethine are administered sequentially.

Also disclosed herein is the method of treating NAFLD, NASH and the other metabolic disorders disclosed herein with plants or biological extracts that contain L-DOPA.

Further disclosed herein is a method of simultaneously treating non-alcoholic steatohepatitis (NASH) or non-alcoholic fatty liver disease (NAFLD) and obesity or another metabolic disorder in a patient which comprises administering to a patient in need of such treatment (i) L-DOPA, or plant or biological extract containing L-DOPA, or a pharmaceutically acceptable salt of L-DOPA within 2 hours of waking and (ii) co-administering with the L-DOPA, or pharmaceutically acceptable salt of L-DOPA or a plant or biological extract containing L-DOPA to the patient in need of such treatment, pantethine with or without, solubilized curcumin.

Further disclosed herein are pharmaceutical formulations comprising one or more agents that increase central dopaminergic neuronal activity and pantethine.

Another embodiment is a pharmaceutical formulation containing one or more agents that increase central dopaminergic neuronal activity, pantethine and solubilized curcumin.

In one example, the solubilized curcumin is in a curcumin solubilization formulation containing phosphatidylcholine, caprylic/capric triglyceride, alcohol, glyceryl stearate, oleic acid, ascorbyl palmitate, and tocopherol.

In another example, the curcumin solubilization formulation contains at least 53% phosphatidylcholine.

In another example, the solubilized curcumin is made into a solid powder with the use of certain excipients such as fumed silica types (e.g., silicon dioxide, SYLOID® XDP (W.R. Grace & Co.) or the like), microcrystalline cellulose and/or others.

Examples of biological sources containing L-DOPA include velvet bean (Mucuna Pruriens), broad bean, and oceanic biological sources.

A still further embodiment comprises a pharmaceutical formulation containing one or more agents that increase central dopaminergic neuronal activity, pantethine, curcumin and a probiotic.

Further embodiments include pharmaceutical formulations comprising L-DOPA including salts or biological sources containing L-DOPA and pantethine; L-DOPA (including salts or biological sources containing L-DOPA) pantethine and curcumin; L-DOPA (including salts or biological sources containing L-DOPA), and L-DOPA (including salts or biological sources containing-DOPA) and pantethine, curcumin and one or more probiotics.

Other features and advantages of the methods and formulations disclosed herein will be apparent from the following detailed description and from the claims.

### DETAILED DESCRIPTION

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As used herein, the terms “about” and “approximately” are defined as being within plus or minus 10% of a given value or state, preferably within plus or minus 5% of said value or state.

As used herein, the term “co-administered” means that one or more drugs are administered at the same time. In some embodiments, the one or more drugs (e.g. dopamine agonist, curcumin and pantethine) are administered sequentially. In some embodiments, the one or more drugs (e.g. pantethine and curcumin) are administered simultaneously.

The present disclosure is based, in part, on the discovery that certain regulators of the central nervous system (CNS) biological clock pacemaker circuit (including but not limited to the suprachiasmatic nuclei (SCN), as well as several other CNS centers) (collectively, the CNS biological clock circuit) are useful in the treatment of both NAFLD and NASH.

The CNS biological clock circuit is the major central nervous system control center in the body for the maintenance of physiological (e.g., metabolic, immunological, behavioral, and reproductive) homeostasis, regulating physiological activities to keep the body in sync with changing (e.g., cyclic) conditions (in, e.g., food availability, predator and prey presence, photoperiod, and season of the year), thereby increasing survivability. The CNS biological clock circuit with the suprachiasmatic nuclei acting a master clock pacemaker for the circadian regulation of whole body physiology both regulates metabolism and enhances repair (including, e.g., immunological) mechanisms in response to tissue damage within the body (for example within the liver as with NASH), both facilitating tissue repair and protecting against (further) tissue damage as a function of its appropriate internal circadian organization.

Key regulators of the CNS biological clock circuit that have been found to be effective in treating NAFLD or NASH include dopamine D1 receptor agonists, dopamine D2 receptor agonists, dopamine D1 agonists plus dopamine D2 receptor agonists, and other agents that increase central dopamine neuronal activity (examples listed below) that when administered within 4 hours of waking in the morning can be effective in treating NASH or NAFLD. Key regulators of the CNS biological clock circuit are known in the art and are described in, e.g., U.S. Pat. Nos. 5,006,526; 5,468,755; 5,565,454; 5,756,513; 5,866,584; 5,877,183; 6,004,972; 6,855,707; and 9,655,865. The disclosures of these patents are incorporated herein by reference.

### Dopamine D1 Receptor Agonists

Dopamine D1 receptor agonists activate or potentiate dopamine D1 receptors or D1-like receptors such as D1 and D2 dopamine receptors. In one embodiment, the dopamine D1 receptor agonist is a selective agonist for the D1 receptor over the D2 receptor (i.e., the compound has a lower K, or EC₅₀ for the D1 receptor than the D2 receptor). In one embodiment, the dopamine D1 receptor agonist is a weak agonist (e.g., K or EC₅₀ of greater than 1 μM or 1 mM) or a partial agonist (i.e., binding affinity less than that of endogenous dopamine for D2 sites) or is not a dopamine D2 receptor agonist (e.g., K or EC₅₀ of greater than 10 mM).

Dopamine D1 receptor agonists that are capable of activating or potentiating D1 dopamine receptors are well known in the art. Specific examples of dopamine D1 receptor agonists include, e.g., dopamine, apomorphine, SKF38393, dihydrexidine, SKF 75670, SKF 82957, SKF 81297, SKF 82958, SKF 82598, A77636, A68930, SKF 82526 (fenoldopam), and racemic trans-10,11-dihydroxy-5,6,6a, 7,8,12b-hexahydro and related benzazepine analogs. Preferred dopamine D1 receptor agonists include SKF38393, dopamine and apomorphine.

One or more dopamine D1 receptor agonists can be administered to a patient to treat NAFLD or NASH, optionally in conjunction with one or more dopamine D2 receptor agonists. The effective dosage will depend on the severity of the disease and the activity of the particular compound(s) employed, and is thus within the ordinary skill of the art to determine for any particular patient. Suitable dosages may be, e.g., in the range of about 0.5 μg to about 1 mg per kg body weight per day for a human patient, or between about 0.7 μg to 0.9 mg per kg of body weight per day. Other suitable dosages may be selected from e.g., 0.1, 0.2, 0.3, 0.4, 0.5, 0.6, 0.7 and 0.8 mg/kg of body weight per day.

### Dopamine D2 Receptor Agonists

Dopamine D2 receptor agonists activate or potentiate dopamine D2 receptors (e.g., D2, D2 short and D2 long receptors). In one embodiment, the D2 receptor agonist is a selective agonist for the D2 receptor over the D1 receptor. In a further embodiment, the D2 receptor agonist is a weak D1 receptor agonist or is not a D1 receptor agonist.

Dopamine D2 receptor agonists are well known in the art. Specific examples of dopamine D2 receptor agonists include, e.g., dopamine, apomorphine, 2-bromo-α-ergocriptine (bromocriptine), terguride, dihydroergotoxine (hydergine), dihydro ergocriptine (DHECP), apomorphine, quinelorane, erfotoxine, 6-methyl-8-carbobenzyloxy-aminoethyl-10-α-ergoline, 8-acylaminoergoline, and 6-methyl-8-α-(N-acyl)amino-9-ergoline.

### Use of Dopamine D1 Plus D2 Receptor Agonists to Treat NAFLD and NASH

One or more dopamine D2 receptor agonists can be administered to a patient to treat NAFLD or NASH, optionally in conjunction with one or more dopamine D1 receptor agonists. It has unexpectedly been discovered that circadian timed administration of dopamine D1 plus D2 receptor agonists to effectuate a circadian peak in dopaminergic activity that mimics or amplifies the circadian peak of such activity in healthy individuals of the same species produces an improvement in NAFLD and NASH that cannot be achieved with either dopamine D1 or D2 receptor agonist treatment alone and the effect on the patients disease can be more than the additive effects of dopamine D1 and D2 receptor agonist treatment separately on the composite of the spectrum of NASH pathologies (e.g., hepatic steatosis, micro and macro-ballooning, fibrosis, inflammation, vascular damage, and cell death). The effective dosage will depend on the severity of the disease and the activity of the particular compound(s) employed, and is thus within the ordinary skill of the art to determine for any particular patient. Suitable dosages of each of D1 and D2 agonist that are effective for treating NAFLD and NASH are, in the range of about 0.5 μg to about 5 mg per kg body weight per day for a human patient and preferably between about 1 and about 2 mg/kg of body weight per day. If a D1 and D2 agonist are administered (or co-administered) to treat NASH or NAFLD, the combined D1+D2 dose should not exceed 5 mg/kg of body weight per day.

Agents that Increase Central Dopamine Neuronal Activity

As used herein, “neuronal activity” refers to either an increase or decrease in the action potential of a neuron. More specifically, as used herein, the term “neuronal activity” refers to either an increase or decrease in the synaptic neurochemical signal transmission of a neuron to another thereby affecting action potential. More narrowly yet, as defined herein, “neuronal activity” refers to the biochemical communication to a (secondary (e.g., post-synaptic)) neuron from either the neurochemical signal transmission of another (primary (e.g., pre-synaptic)) neuron (e.g., as via an endogenous neurotransmitter) or from any neuromodulatory compound (e.g., an exogenous neurotransmitter receptor modulator such as a pharmaceutical agent) thereby affecting action potential or neurotransmitter release of the secondary neuron. As such, an increase in dopaminergic neuronal activity would be characterized by (a) an increase in release of dopamine molecules from a dopamine producing (primary) neuron, an increase in dopamine molecules within the synapse by any mechanism, and/or increase in dopamine-mimetic compound(s) from any source (e.g., pharmaceutical) resulting in increased binding to dopaminergic receptor sites of other (secondary) neuron(s) that affect said other neuron(s)′ action potential or neurotransmitter release in a manner consistent with increased dopamine ligand-dopamine receptor binding signal transduction (e.g., post-synaptic dopamine receptor agonist) and/or (b) an increase in sensitivity or responsiveness of said “other (secondary)” neuron(s) to such dopamine or dopamine-mimetic compound(s)′ ability to affect action potential or neurotransmitter release in said “other (secondary)” neuron (e.g., as an increase in dopamine receptor number or affinity or responsiveness For the sake of clarity, post-synaptic dopamine receptor agonists include dopamine D1, D2, D3, D4, and D5 receptor agonists.

Agents that increase central dopamine neuronal activity include, e.g., specific dopamine reuptake inhibitors, dopamine presynaptic transporter inhibitors (e.g., GBR-12909, GBR-12935, and 1-[2-(diphenylmethoxy)ethyl]-4-[(2E)-3-phenyl-2-propen-1-yl]piperazine (GBR-12783), dopamine presynaptic auto receptor antagonists (e.g., AJ76), presynaptic dopamine release enhancers (oleyethanolamide and its analogs, amphetamine, cocaine), post synaptic dopamine receptor agonists such as dopamine D1 and D2 receptor agonists (optionally used in conjunction with compounds that circumvent agonist-induced decreases in synaptic dopamine levels), (dopamine D1 and D2 receptor agonists-see below), dopamine synthesis stimulators (tyrosine hydroxylase stimulators, phenylaminotetralins), and dopamine catabolisminhibitors (monooxygenase inhibitors). Agents that increase central dopamine neuronal activity also include, e.g., dopamine/norepinephrine reuptake inhibitors (e.g., cocaine, bupropion, mazindol, nomifensine, tesofensine, and methylphenidate) used in conjunction with one or more agents that reduce norepinephrine activity. Also agents that inhibit dopamine beta hydroxylase (DBH) such as pantethine are agents that may increase central dopamine neuronal activity.

Specific examples of agents that increase central dopamine neuronal activity include, e.g., 1-[2-(diphenylmethoxy)ethyl]-4-(3-phenylpropyl) piperazine (GBR-12935); brain derived neurotrophic factor (BDNF); (4aR-trans)-4,4-a,5,6,7,8,8a,9-octahydro-5-propyl-1H-pyrazolo[3,4-g]quinoline (quinpirole); quinelorane; 1-phenyl-7,8-dihydroxy-2,3,4,5-tetrahydro-1H-3-benzazepine hydrochloride (SKF38393); Deprenyl (selegiline); apomorphine; pramipexole (Mirapex®); 1-2-(bis(4-fluorophenyl)-methoxy)-ethyl-4-(3-phenylpropyl) piperazine (GBR-12909 or vanoxerine); dihydroergotoxine (hydergine); dihydro-alpha-ergocriptine; bromocriptine: lisuride; terguride; methylphenidate; bupropion; nomefensine; phenylaminotetralins; talexipole; oleylethanolamide and its analogs; dopamine, and L-DOPA, or a biological source containing L-DOPA (e.g., broad bean or velvet bean (Mucuna pruriens) or extracts thereof.

L-DOPA (levodopa, 3,4-dihydroxyphenylalanine) is an amino acid synthesized in the brain by l-tyrosine (also an amino acid) and is a precursor to the neurotransmitter dopamine. Bromocriptine and L-DOPA are preferred agents that increase central dopamine neuronal activity for use in the methods and formulations disclosed herein. The L-DOPA is preferably plant or biological source derived L-DOPA. Plant derived L-DOPA is commercially available in plant extracts from Mucuna pruriens (velvet bean) (available from e.g. Double Wood LLC, Philadelphia, PA), and Vicia faba (broad bean) Vigna aconitifolia, Vigna unguiculata, Vigna vexillata, Prosopis chilensis, Pileostigma malabarica, Phanera vahlis, Parkinsonia acculeata, Macuna urens, Canvavalia glandiata, Cassia floribanda, Casia hirsute and Dalbergia retusa, available from several different manufacturers (e.g., Swanson Health Products PO BOX 2803 Fargo, ND, and Vitacost 5400 Broken Sound Blvd., suite 500, Boca Raton, FL 33487).

One or more agents that increase central dopamine neuronal activity can be administered alone to a patient to treat NAFLD or NASH, optionally in conjunction with one or more dopamine D1 receptor agonists and/or dopamine D2 receptor agonists. The effective dosage will depend on the severity of the disease and the activity of the particular compound(s) employed, and is thus within the ordinary skill of the art to determine for any particular patient. Suitable dosages may be, e.g., in the range of about 0.5 μg to about 5 mg per kg body weight per day for a human patient, and more preferably from about 0.7 μg to about 3 mg per kg body weight per day. Suitable dosages may preferably be, e.g., in the range of about 2 μg to about 1 mg per kg body weight per day for a human patient. The dosage range for the dopamine D1 agonists can be from 0.5 μg to about 5 mg per kg body weight per day. The dosage range for the dopamine D2 agonists can be from 0.5 μg to about 5 mg per kg body weight per day. In no event should a patient receive a combined total dose of D1 and D2 that is more than 5 mg/kg of body weight per day of D1 and/or D2, i.e. 5 mg/kg body weight per day is the maximum combined dose of D1 and D2 e.g. 2 mg/kg D1 and 3 mg/kg D2 per day.

An acceptable method to reinstate the circadian peak of central dopaminergic neuronal activity in a subject with a diminished circadian peak of central dopaminergic neuronal activity that is superior to treatment with a dopamine receptor agonist is by treatment with a combination of dopamine receptor agonist, pantethine, and curcumin or L-DOPA (e.g., within a biological source or otherwise), pantethine and curcumin within 4 hours of waking from the daily sleep cycle.

### Use of Dopamine Neuronal Activity Enhancers Plus Gastrointestinal Anti-Inflammatory Agents to Treat NAFLD and NASH

This disclosure additionally relates to methods for timed administration of a combination of compositions comprising one or more dopamine neuronal activity enhancers (e.g., dopamine receptor agonists) and one or more agents that reduce pro-inflammatory microbiota (e.g., gastrointestinal-targeted antibiotic agents) for the treatment of non-alcoholic steatohepatitis (NASH) and non-alcoholic fatty liver disease (NAFLD or hepatic steatosis).

It has been discovered that the impact of administering appropriately circadian timed and formulated dopamine agonist therapy that mimics the circadian peak in central dopaminergic neuronal activity of a healthy individual of the same species and sex to reduce NAFLD and NASH can be amplified by administration of agents that reduce a pro-inflammatory gastrointestinal (GI) tract environment (including microbiota) (e.g., administration of prebiotics, antibiotics and/or probiotics (collectively termed gastrointestinal pro-health agents; abbreviated as GPH agents)) to produce an interaction between the two therapies leading to an attenuation or reversal of disease symptoms. Appropriate circadian timed administration of such GPH agents can further enhance their efficacy and therapeutic index (ratio of efficacy to adverse effects). The full breadth of beneficial health effects resulting from the combination therapy (circadian timed dopamine neuronal activation plus circadian timed administration of GPH agents) on the NAFLD/NASH disease states cannot be accomplished by either therapy alone and can be more than the additive effects of each therapy. The optimal circadian time for such dopamine neuronal activation, pantethine, solubilized curcumin, and GPH administration is within 4 hours and preferably within 2 hours of morning waking from daily sleep or between 0400 and 1200 hours of the day.

Prebiotics are one class of GPH agents. Prebiotics are defined as dietary supplements and/or food sources such as non-digestible nutrients and polysaccharides or others that stimulate the selective growth of microbial genus/species in the gut microbiota that confer health benefits or that reduce the pro-inflammatory environment of the GI tract or that improve peripheral glucose or lipid metabolism, e.g., non-digestible carbohydrates such as inulin-type fructans, and the galacto-oligosaccharides, xylans including arabinoxylans, chiton glucans, polyphenols, oligofructose, pectins, hemicellulose, pantethine, resistant starch, solubilized curcumin, short chain fatty acids (up to C12 chain length) oleylethanolamide and its analogs). Fungi and edible mushroom agents can also be a source of such prebiotics.

It has been found that a solubilized form of curcumin provides surprisingly improved treatment of NASH and NAFLD when administered with pantethine and with or without a dopaminergic neuronal activity enhancer. In one preferred embodiment, the solubilized curcumin is in a curcumin solubilization formulation containing phosphatidylcholine, caprylic/capric triglyceride, alcohol, glyceryl stearate, oleic acid, ascorbyl palmitate, and tocopherol. In another embodiment this formulation contains at least 53% phosphatidylcholine. The formulation constituents help to stabilize the curcumin, render it more absorbable and improve its activity. Solubilized curcumin is one preferred prebiotic for use in the methods and formulations described herein. In general the solubilized formulation can be prepared in solution with curcumin, a phospholipid, a triglyceride, one or more fatty acids, a fat soluble form of ascorbic acid and one or more alcohols including fat soluble alcohols and also including tocopherol. Furthermore, the solubilized curcumin can then be made into a powder by addition of certain excipients such as fumed silica types (e.g., silicon dioxide or SYLOIDE®) and microcrystalline cellulose and others suitable for solid dosage form preparations for oral administration.

Pantethine is another preferred prebiotic for use in the methods of treatment and pharmaceutical formulations disclosed herein. Pantethine is administered at a dose of between about 1 mg/kg to about 0.15 g/kg of body weight per day and preferably between about 5 mg/kg to about 0.1 g/kg of body weight per day. Curcumin that has been solubilized as described herein is another preferred prebiotic for use in the methods of treatment and pharmaceutical formulations disclosed herein and may be administered in the dosage range from about 0.1 mg/kg to about 0.07 g/kg of body weight per day and preferably from about 2 mg/kg to about 0.03 g/kg of body weight per day. Combined administration of solubilized curcumin and pantethine along with a dopaminergic neuronal activity enhancer (preferably bromocriptine citrate or L-DOPA or a biological source containing L-DOPA such as mucuna pruriens) has been found to be especially effective in treating NASH and NAFLD. In one embodiment, the dopaminergic neuronal activity enhancer may be formulated in one solid dosage form (for example as a pill, tablet or capsule) and the pantethine and solubilized curcumin are formulated in a separate solid dosage form that is usually co-administered (either sequentially or simultaneously) with the dopaminergic neuronal activity enhancer dose. The prebiotics are preferably administered within 4 hours (and preferably within 2 hours) of waking from the daily sleep cycle (between 0400 and 1200 hours).

Probiotics are another class of GPH agents. Probiotics are defined by the World Health Organization as live microorganisms which, when administered in adequate amounts, confer a beneficial health effect on the host. Administration of probiotics (for example such as Proteobacteria, Lactobacillus gasseri, Lactobacillus spp., bacilli, clostridium clusters IV and XVI, Akkermansia muciniphila, L. caesi, L. plantarum, bifidobacteria) in combination with circadian timed agents that increase central dopaminergic neuronal activity produce a beneficial effect on NAFLD and NASH that cannot be achieved with either therapeutic alone and is greater than the additive effects of each therapeutic. These agents can be administered at a dose of between about 10 mg to about 5 grams per day.

A preferred probiotic for use in the disclosed formulations and methods is Proteobacteria or strains of Lactobacillus including but not limited to Lactobacillus gasseri, Lactobacillus spp., bacilli, acidophilus, bulgaricus, cassei, clostridium clusters IV and XVI, Akkermansia muciniphila, L. caesi, L. plantarum, a variety of bifidobacterial strains including bifidum, lactis, longum.

Antibiotics are another class of GPH agent. Antibiotics are xenobiotics that have anti-bacterial activity (bactericidal or bacteriostatic). A beneficial effect of GPH agents such as antibiotics on NAFLD/NASH, dysmetabolism, and gastrointestinal pro-inflammatory disease, resides in part in their ability to improve (increase) the ratio of beneficial versus adverse health bacteria in the GI tract as well known to those skilled in the art (e.g., antibiotics increase beneficial bacteria such as Proteobacteria, a variety of Lactobacilus strains including but not limited to Lactobacillus gasseri, Lactobacillus spp., bacilli, acidophilus, bulgaricus, cassei, clostridium clusters IV and XVI, Akkermansia muciniphila, L. caesi, L. plantarum, a variety of bifidobacterial strains including bifidum, lactis, longum) or decrease adverse bacteria such as Staphylococcus aureus, and enterobacteriaceae. GPHs also include molecules that influence the metabolism of ingested nutrients to generate gastrointestinal hormonal release that favors a healthy metabolism such as GLP-1 and peptide YY and numerous metabolized nutrients such as short chain fatty acids (for example C2-C14 free fatty acids such as acetic, propionic and butyric acids) that provide a metabolic and immune health benefit Effectiveness of such GPH agents can be enhanced by the appropriate circadian timing of such GPH agents. Suitable gut-targeted antibiotics for use in practicing the methods disclosed herein include, by way of non-limiting example, rifampin-available from MMX® technology (Cosmo Pharmaceuticals, Dublin, Ireland) for delivery to the lower GI tract, gut-targeted ampicillin, neomycin, vancomycin, and metronidazole. These agents can be administered at a dose of between 0.1 mg to 100 mg per kg body weight per day. The antibiotics are preferably co-administered with the dopaminergic or neuronal activity enhancers.

When used alone to treat NAFLD or NASH, GPH agents that reduce pro-inflammatory microbiota do not produce clinically significant reversal of disease symptoms. Therefore, a different approach to treatment of these diseases is required. It has been found that these agents are most effective if co-administered with agents that increase the circadian peak in central dopaminergic neuronal activity such as for example circadian-timed bromocriptine, a pharmaceutically acceptable salt of bromocriptine or L-DOPA, or a biological source containing L-DOPA (e.g., broad bean or velvet bean (Mucuna pruriens) or extracts thereof.

Furthermore, long term use of antibiotics as a means of reducing the number of pro-inflammatory gut microbiota of the patient is not a practical means of treating NASH or NAFLD inasmuch as such action can lead to the development of antibiotic-resistant strains of bacteria in the individual, and can also lead to eventual damage of the healthy gut microbiota by disrupting the normal “healthy” relative ratios of various bacteria within the gastrointestinal tract. The beneficial effect of some of the central dopamine neuronal activity increasing therapies on NAFLD or NASH may best be achieved by their parenteral administration in a formulation and manner that effectuates an increase in central dopaminergic neuronal activity that mimics or approximates the circadian peak in central dopaminergic neuronal activity of a healthy individual of the same species and sex. Such dopamine neuronal activity enhancers that are administered in a manner that results in a circadian rhythm of central dopaminergic activity that mimics that of a healthy individual of the same species and sex are most beneficial for treatment of NAFLD and NASH.

Using the formulations and methods described herein it is possible to effectuate improvements in several metabolic pathologies including by way of non-limiting example type 2 diabetes, prediabetes, cardiovascular disorders, atherosclerosis, arteriosclerosis, heart failure, cerebrovascular disease, coronary artery disease, peripheral vascular disease, hypertension, high cholesterol, renal disease, hyperglycemia, fatty liver, and/or NASH by potentially acting both centrally and at specific downstream peripheral targets such as biochemical pathways within the GI tract, liver or muscle.

### Pharmaceutical Compositions

In some aspects, the methods described herein include the manufacture and use of pharmaceutical compositions and medicaments that include the compounds disclosed herein. Also included are the pharmaceutical formulations containing such compositions.

One preferred composition for treating NASH or NAFLD contains bromocriptine or a pharmaceutically acceptable salt of bromocriptine such as bromocriptine citrate, pantethine and solubilized curcumin. Another preferred composition for treating NASH or NAFLD contains L-L-DOPA, or a biological source containing L-DOPA (e.g., broad bean or velvet bean (Mucuna pruriens) or extracts thereof, pantethine with or without solubilized curcumin. These compositions are also effective to simultaneously treat obesity in patients being treated for NASH or NAFLD. Thus, for example, the combination of bromocriptine or a pharmaceutically acceptable salt of bromocriptine such as bromocriptine citrate, pantethine and solubilized curcumin is useful to treat a patient's obesity simultaneously with treatment of the patient's NASH or NAFLD with the methods disclosed herein.

Obesity is determined in several different ways. One preferred method used by the Centers for Disease Control (CDC) and the World Health Organization is based on Body Mass Index (patient's weight in kilograms divided by their height squared in meters.) Patients with BMI over 30.0 are considered obese. Additionally, there are several devices that determine body fat store levels such as DEXA scan, electro-impedance, and MRI. DEXA is a noninvasive body composition test that employs two X-ray beams sent through the body at different energy levels that is used to measure body fat. The DEXA results include graphs that show trends over time, in particular, how percent body fat has changed over time and how body fat mass compares to lean mass trends over time.

In some aspects, the compositions disclosed herein can include other compounds, drugs, or agents used for the treatment of liver disease (e.g., NAFLD or NASH). For example, in some instances, pharmaceutical compositions disclosed herein can be combined with one or more (e.g., one, two, three, four, five, or less than ten) compounds. Such additional compounds can include, e.g., NAFLD drugs known in the art (e.g., pioglitazone) and GLP-1 receptor agonists (e.g. liraglutide, semaglutide) and SGLT2 inhibitors (e.g., empagliflozin, dapagliflozin). When co-administered, the compounds disclosed herein can operate in conjunction with NASH or NAFLD drugs known in the art to produce mechanistically additional, additive or synergistic therapeutic effects.

In some aspects, the pH of the compositions disclosed herein can be adjusted with pharmaceutically acceptable acids, bases, or buffers to enhance the stability of the compositions or their delivery form.

Pharmaceutical compositions typically include a pharmaceutically acceptable carrier, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable carrier, adjuvant, or vehicle is a composition that can be administered to a patient, together with a compound of the invention, and which does not destroy the pharmacological activity thereof and is nontoxic when administered in doses sufficient to deliver a therapeutic amount of the compound. Exemplary conventional, non-toxic pharmaceutically acceptable carriers, adjuvants, and vehicles well known in the art include saline, solvents, dispersion media, coatings, antibacterial and antifungal agents such as isotonic and absorption delaying agents, and the like, compatible with pharmaceutical administration to humans. Particularly important carriers for the present disclosed compositions are disintegrating agents such as Pharmaburst® (ABF Ingredients) or Explotab® (JRS Pharma) and binders such as microcrystalline celluloses or fumed silicas (e.g., silicon dioxide or SYLOID®).

In particular, pharmaceutically acceptable carriers, adjuvants, and vehicles that can be used in the pharmaceutical formulations disclosed herein include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

As used herein, the compounds disclosed herein are defined to include pharmaceutically acceptable derivatives or prodrugs thereof.

As used herein the term “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a mammal (e.g., by allowing an orally administered compound to be more readily absorbed into the blood) or which enhance delivery of the parent compound to a biological compartment (e.g., the brain or lymphatic system) relative to the parent species.

As used herein, the term “prodrug” means a pharmacologically inactive substance that is converted in the body (such as by enzymatic action) into a pharmacologically active drug. Preferred prodrugs include derivatives where a group that enhances aqueous solubility or active transport through the gut membrane is appended to the structure of formulae described herein. Such derivatives are recognizable to those skilled in the art without undue experimentation. Reference is expressly made to the teachings of Burger's Medicinal Chemistry and Drug Discovery, 5th Edition, Vol. 1, Principles and Practice. Examples of prodrugs useful as acting as dopamine neuronal activity enhancers include analogs of L-DOPA including, L-DOPA methyl ester, dimeric L-DOPA diacetyl esters, d-phenylglycine L-DOPA, diamides of (O, O-diacetyl)-L-DOPA-methyl ester, and dendritic L-DOPA dendrimer prodrugs (HO-G2-NH2).

The compounds disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivative thereof.

In particular, pharmaceutically acceptable salts of the compounds disclosed herein include, e.g., those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, mesylate, methane sulfonate, 2-naphthalenesulfonate, nicotinate, nitrate, palmitate, phosphate, picrate, pivalate, propionate, salicylate, succinate, sulfate, tartrate, tosylate, trifluoromethylsulfonate, and undecanoate. The citrate salt of bromocriptine is one preferred salt for use in the methods of treatment and formulations described herein. Salts derived from appropriate bases include, e.g., alkali metal (e.g., sodium), alkaline earth metal (e.g., magnesium), ammonium and N-(alkyl) 4+ salts. The invention also envisions the quaternization of any basic nitrogen-containing groups of the compounds disclosed herein. Water or oil-soluble or dispersible products can be obtained by such quaternization.

In some aspects, the pharmaceutical compositions disclosed herein can include an effective amount of one or more of the compounds disclosed herein. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment or prevention of NAFLD or NASH). In some aspects, pharmaceutical compositions can further include one or more additional compounds, drugs, or agents used for the treatment of NAFLD in amounts effective for causing an intended effect or physiological outcome (e.g., treatment or prevention of NAFLD).

In some aspects, the pharmaceutical compositions disclosed herein can be formulated for sale in the United States, import into the United States, or export from the United States.

### Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/FormsSubmissionRequirements/Electr onicSubmissions/DataStandardsManualmonographs). In particular, the pharmaceutical compositions can be formulated for and administered via oral, parenteral, or transdermal delivery. The term “parenteral” as used herein includes transmucosal, subcutaneous, sublingual, intracutaneous, intravenous, intramuscular, intraperitoneal, intra-articular, intra-arterial, intrasynovial, intrasternal, intrathecal, intralesional, and intracranial injection or infusion techniques that result in absorption of the active agent into the body by means that is primarily outside of (without) the gastrointestinal tract.

For example, the pharmaceutical compositions disclosed herein can be administered, e.g., parenterally, topically, rectally, nasally (e.g., by inhalation spray or nebulizer), buccally, sublingually, vaginally, sub dermally (e.g., by injection or via an implanted reservoir), or ophthalmically. Parenteral administration is the preferred embodiment for administration of the neuronal activity enhancers employed in the methods of treatment disclosed herein.

For example, pharmaceutical compositions of this invention can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with conventional emulsifying or suspending agents. If desired, certain sweetening, flavoring, or coloring agents can be added.

### Solubilized Curcumin

In the case of oral administration of curcumin it has been found that the solubility of curcumin is critical to enhancing its bioavailability and consequently its bioactivity. Curcumin is very insoluble in water and difficult to solubilize in organic liquids that are both safe and enhance the agent's bioavailability and bioactivity. In this connection it has been found that the solubility and bioactivity of the compound can be greatly enhanced by solubilization of the curcumin in phosphatidylcholine or lysophosphatidylcholine with or without ethanol, water, mono- or diglycerides, oleic acid, caprylic/capric triglycerides, alpha tocopherol, ascorbyl palmitate, glyceryl stearate, soy acid, safflower seed oil, or propylene glycol. A particularly useful curcumin solubilization formulation is phosphatidylcholine (not less than 53%), caprylic/capric triglyceride, alcohol, glyceryl stearate, oleic acid, ascorbyl palmitate, and tocopherol

Curcumin is also somewhat unstable in pharmaceutical compositions. To assist in stabilizing the solubilized curcumin, it is desirable to include a stabilizer in the formulation. In some embodiments, the stabilizer is a vegetable oil. Examples of vegetable oils include almond oil, apricot kernel oil, canola oil, castor oil, cinnamon oil, coconut oil, corn oil, cottonseed oil, flaxseed oil, palm oil, palm kernel oil, olive oil, peanut oil, rapeseed oil, safflower oil, soybean oil and sunflower oil and mixtures thereof

The following preferred process for solubilization of curcumin provides a product having improved oral administration properties and superior gastrointestinal absorption. 700 mg of curcumin are added to a mixture containing 4 ml of phosphatidylcholine (of not less than 53% of total solution volume), between 3-6% of total volume of ethanol, up to 6% of total volume of lysophosphatidylcholine, and a mixture of caprylic/capric triglycerides, glyceryl stearate, oleic acid, ascorbyl palmitate, and DL-alpha-tocopherol each in amount of between 0.1 to about 40% (preferably between about 1 and about 15%) of total volume. The curcumin-phosphatidylcholine mixture is stirred until clear while being heated to between 50° C. to 60° C. Optionally add up to 10 ml of water and stir until solution is homogeneous. Pantethine is prepared in the same solubilization solution as curcumin or in water, preferably at a concentration of 0.1 to 5 grams per ml.

These formulations can be prepared by combining one or more of the biologically active compounds disclosed herein with a suitable non-irritating excipient which is solid at room temperature but liquid at the rectal temperature and therefore will melt in the rectum to release the active components. Such materials include, but are not limited to, cocoa butter, beeswax, and polyethylene glycols.

Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

In another embodiment, the pharmaceutical compositions disclosed herein can be prepared as sterile pharmaceutical formulations for administration by injection (e.g., as a solution or powder). Pharmaceutical formulations containing such compositions can be prepared according to techniques known in the art using suitable dispersing or wetting agents (such as, for example, Tween 80 (polysorbate 80)) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables and oral formulations, as are natural pharmaceutically-acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

The additional agents can be administered separately, as part of a multiple dose regimen, from the compounds of this invention. In some instances, those agents can be part of a single dosage form, mixed together with the compounds of this invention in a single composition.

In some aspects, the pharmaceutical compositions disclosed herein can be included in a container, pack, or dispenser together with instructions for administration.

### Methods of Treatment

The methods disclosed herein contemplate administration of an effective amount of a compound or composition to achieve the desired or stated effect. Typically, the compounds or compositions of the present method will be administered at the appropriate dosage, within an appropriate pharmaceutical or nutraceutical formulation and at the appropriate time of day so that levels of dopaminergic activity are not elevated throughout the greater portion of the day but are confined to a discrete daily interval of the day that approximates the natural daily circadian peak of central nervous system dopaminergic activity in healthy individuals of the same species and sex (i.e., individuals without NAFLD, NASH, metabolic disorders or increased levels of metabolic or non-metabolic biomarkers of NAFLD or NASH or metabolic disorders).

In particular, the compounds or compositions of the invention can be administered in formulations either within 4 hours after waking from the daily sleep cycle or between 0400 and 1200 hours:
1. To create a peak of CNS dopaminergic activity between 0400 and 1200 hours of the day,
2. To create a peak of CNS dopaminergic activity within 4 hours (preferably 2 hours) of waking (defined as the onset of daily locomotor activity (i.e., in the morning for diurnal species such as humans)
3. To create a plasma peak of dopaminergic neuronal activity stimulating agent(s) between 0400 and 1200 hours of the day, or
4. To create a plasma peak of dopaminergic neuronal activity stimulating agent(s) within 4 hours (preferably 2 hours) of waking (defined as the onset of daily locomotor activity i.e., in the morning for diurnal species such as humans).

Such administration can be used as a chronic or acute therapy. The amount of active ingredient that can be combined with the carrier materials to produce a single dosage form will vary depending upon the host treated and the particular mode of administration. A typical preparation will contain from about 0.1% to about 99% active compound (w/w). In another embodiment, such preparations can contain from about 1% to about 90% active compound.

In some aspects, the present disclosure provides methods for using a composition, including pharmaceutical compositions (indicated below as ‘X’) disclosed herein in the following methods:

Substance X for use as a medicament in the treatment of one or more diseases or conditions disclosed herein (e.g., NASH, referred to in the following examples as ‘Y’). Use of substance X for the manufacture of a medicament for the treatment of Y; and substance X for use in the treatment of Y.

In some aspects, the methods disclosed include the administration of a therapeutically effective amount of one or more of the compounds or compositions described herein to a subject (e.g., a mammalian subject, e g, a human subject) who is in need of, or who has been determined to be in need of, such treatment. In some aspects, the methods disclosed include selecting a subject and administering to the subject an effective amount of one or more of the compounds or compositions described herein, and optionally repeating administration as required for the prevention or treatment of NAFLD or NASH.

In some aspects, subject selection can include obtaining a tissue sample including a plasma sample or a liver biopsy from a subject (e.g., a candidate subject) and testing the sample for existence of NASH. In some aspects, the NASH subject can be confirmed or identified, e.g. by a healthcare professional, as having had or having a condition or disease by use of medical devices such as MRI or CT scan or biomolecule radioisotope detection within liver indicative of liver inflammation and fibrosis. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells, or tissue.

In some aspects, methods of treatment can include multiple administrations, and repeating administration of one or more compounds disclosed herein on a daily basis as required for the prevention or treatment of NASH. In some aspects, methods of treatment can include assessing a level of disease in the subject prior to treatment, during treatment, or after treatment. In some aspects, treatment can continue until a decrease in the level of disease in the subject is detected. In some aspects, the treatment can continue to abate the onset or progression of NAFLD or NASH.

The term “subject,” or “patient” as used herein, refers to any animal. In some instances, the subject or patient is a mammal. In some instances, the term “subject,” or “patient” as used herein, refers to a human (e.g., a man, a woman, or a child).

The terms “administer”, “administering”, or “administration” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating” or “treatment” as used herein, refer to partially or completely alleviating, inhibiting, ameliorating, or relieving the disease or condition from which the subject is suffering. This means any manner in which one or more of the symptoms of a disease or disorder (e.g., NAFLD or NASH or obesity or a metabolic disorder) are ameliorated or otherwise beneficially altered such as reducing the rate of progression of the disease, halting the progression of the disease, or reversing the disease progression. As used herein, amelioration of the symptoms of a particular disorder (e.g., NAFLD or NASH) refers to any lessening, whether permanent or temporary, lasting or transient that can be attributed to or associated with treatment by the compositions and methods of the present invention. In some embodiments, treatment can promote or result in, e.g., reductions in one or more symptoms associated with NAFLD or NASH in a subject relative to the subject's symptoms prior to treatment. In some embodiments, treatment can produce a reduction of the progression (or reversal of pathologic etiological factors) of NAFLD or NASH. In some embodiments, treatment can repair damage to the liver in subjects with NASH. In some embodiments, treatment can reverse and/or reduce progression of NAFLD to NASH. In some embodiments, treatment can reduce the level or severity of NAFLD. In some embodiments, treatment can reduce the progression of NASH in a subject with NASH.

In particular, treatment as disclosed herein can, e.g.:
1. Reverse (repair) and/or reduce progression or progression rate of hepatic lobular ballooning from NASH.
2. Reverse (repair) and/or reduce progression or progression rate of hepatic fibrosis from NASH.
3. Reverse (repair) and/or reduce progression or progression rate of hepatocellular apoptosis or necrosis from NASH.
4. Reverse (repair) and/or reduce progression or progression rate of liver immune dysfunction from NASH.
5. Reverse (repair) and/or reduce progression or progression rate of hepatic lobular inflammation from NASH
6. Reverse (repair) and/or reduce progression or progression rate of hepatic macro vesicular steatosis from NASH.
7. Reduce the severity of NAFLD (i.e., reduce the liver fat content in an individual with NAFLD).
8. Reduce the severity of fatty liver and other metabolic disorders
9. Reduce the severity of NASH and metabolic disorders
10. Delay or retard the onset of NAFLD.

The terms “prevent”, “preventing”, and “prevention” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention.

As used herein, the term “preventing a disease” (e.g., preventing NAFLD or NASH) in a subject means for example, to stop the development of one or more symptoms of a disease in a subject before they occur or are detectable, e.g., by the patient or the patient's doctor. Preferably, the disease (e.g., NAFLD or NASH) does not develop at all, i.e., no symptoms of the disease are detectable. However, it can also result in delaying or slowing of the development of one or more symptoms of the disease. Alternatively, or in addition, it can result in the decreasing of the severity of one or more subsequently developed symptoms.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present. Dosages applicable for the methods described herein include for example 1 to 20 mg/kg body weight L-DOPA (e.g., 6 to 150 mg/kg Mucuna pruriens), 5 to 100 mg/kg pantethine, plus 2 to 30 mg/kg curcumin or 0.7 to 900 ug/kg agents that increase central dopaminergic neuronal activity, 5 to 100 mg/kg pantethine, plus 2 to 30 mg/kg curcumin.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

The use of GPH agents that act as antibiotics that reduce a pro-inflammatory microbiota may be terminated after several months of use so as to avoid development of resistant bacteria strains, while the dopamine affecting agents are continued longer term.

The mammalian microbiome of the gastrointestinal (GI) tract exhibits circadian rhythms of biological activity, is responsive to circadian rhythm modulation by neural and humoral factors from the host, and can produce molecules that influence the central circadian pacemaker system. It has now been discovered that the time of day at which GPH agents are administered influences their ability to reduce the symptoms of NAFLD and NASH. A particularly effective time to administer GPH agents is at the onset of daily locomotor activity (i.e. in the morning, within about four, and preferably within about two, hours of awakening from the daily sleep cycle). When using agents that increase central dopaminergic neuronal activity or central dopaminergic to noradrenergic neuronal activity in conjunction with such GPH agents to treat NAFLD or NASH, the optimal treatment regimen is to administer the GPH agents at the onset of daily locomotor activity and to administer the agents that increase central dopaminergic neuronal activity or central dopaminergic to noradrenergic neuronal activity in a manner that effectuates a peak in central circadian dopaminergic neuronal activity at the same time of day that the central circadian peak of dopaminergic neuronal activity occurs in a healthy individual of the same species—this is at or near the onset of locomotor activity or within about four hours before and four hours (preferably 2 hours) after awakening. The dopaminergic agonists are best administered parenterally (so that the principal route of drug absorption of the agents is not through the gastrointestinal tract). Parenteral administration avoids the loss of potency resulting from first pass metabolism in which the active pharmaceutical agent is metabolized in the patient's liver.

The term “parenteral administration” is defined herein to mean administration to a patient in a manner that provides for the absorption of a substantial amount of the drug through other than the gastric and/or intestinal mucosa of the GI tract.

Parenteral administration also reduces adverse effects of the dopamine agonists such as nausea, vomiting and gastrointestinal discomfort and is a preferred route of administration of such agents.

A particularly effective combination of dopaminergic and GPH agents for treatment of all the disorders described herein is circadian timed administration of dopamine receptor agonist (preferably within 2 hours of waking in the morning) (e.g., D2 receptor agonist (e.g., bromocriptine citrate) and/or D1 agonist) plus solubilized curcumin and pantethine, or circadian timed administration of dopamine D1 and/or D2 receptor agonist(s) plus circadian timed administration of pantethine and curcumin or combinations thereof plus probiotics. Another effective combination of dopaminergic and GPH agents for all the purposes described herein is circadian timed administration of L-DOPA, or a biological source containing L-DOPA (e.g., broad bean or velvet bean (Mucuna pruriens) or extracts thereof, solubilized curcumin and pantethine with a probiotic. In some embodiments a probiotic such as Lactobaccilus or bifidobacteria strains is co-administered with the other constituents. The optimal time for administration (circadian timing) of the above agents is one that effectuates a circadian peak in dopaminergic activity that mimics or amplifies the natural circadian peak of dopaminergic activity observed in a healthy individual of the same species (e.g., within about 4 hours and preferably within 2 hours of the daily waking time from the daily sleep cycle). However, the probiotic in these combinations also may be administered at any time during the day and remain effective treatment of NAFLD or NASH. Diagnosis of NAFLD or NASH in the individual by established serological, radiological, biochemical, and/or histological methods of examination of the liver (including from liver biopsy). Administration of one or more agents that increase central dopaminergic neuronal activity (e.g., agents selected from the group consisting of L-DOPA, neuronal dopamine synthesis stimulators such as BDNF, neurotrophic growth factor, cerebral dopamine neurotrophic factor, oleoylethanolamide, phenylaminotetralins, neuronal postsynaptic dopaminergic receptor stimulators such as dopamine D2 receptor agonists bromocriptine, dihydro ergocriptine, dihydroergotoxine (hydergine), quinelorane Dopamine D1 receptor agonists benzazepine, SKF38393, SKF82958, neuronal presynaptic dopamine reuptake inhibitors such as bupropion, methylphenidate, nomifensine, mazindol, cocaine, neuronal presynaptic dopamine auto receptor blockers such as AJ76, neuronal presynaptic dopamine release enhancers such as amphetamine, adenosine, dopamine degradation enzyme inhibitors such as monooxygenase inhibitors, neuronal postsynaptic dopamine signal transduction stimulators such as DARPP-32, and combinations thereof including dopamine D₁ or D₂ receptor agonists) to effectuate in the patient a daily peak in the circadian rhythm of central dopaminergic neuronal activity that approximates the daily peak phase of the circadian rhythm of such activity observed in healthy individuals (i.e., provides a peak in central dopaminergic neuronal activity at approximately the same time of day that such activity peaks in healthy individuals (preferably within about 2 to 4 hours of the morning waking time from daily sleep)) in a dose range of about 0.05 ug to about 5 mg/kg body weight/day for a period sufficient to observe reversal of disease state progression (about 1 to 24 months). Specific examples of dopamine D2 receptor agonists include, e.g., 2-bromo-α-ergocriptine (bromocriptine), terguride, dihydroergotoxine (hydergine), dihydro ergocriptine (DHEC), erfotoxine, 6-methyl-8-carbobenzyloxy-aminoethyl-10-α-ergoline, 8-acylaminoergoline, and 6-methyl-8-α-(N-acyl)amino-9-ergoline, apomorphine, dopamine, and quinelorane. One preferred dopamine D2 receptor agonist is the citrate salt of bromocriptine. Specific examples of dopamine D1 receptor agonists include, e.g., dopamine, apomorphine, SKF38393, dihydrexidine, SKF 75670, SKF 82957, SKF 81297, SKF 82958, SKF 82598, A77636, A68930, SKF 82526 (fenoldopam), and racemic trans-10,11-dihydroxy-5,6,6a, 7,8,12b-hexahydro and related benzazepine analogs. A preferred dopamine D1 receptor agonist is SKF38393 or apomorphine. One or more dopamine D1 receptor agonists can be administered to a patient to treat NAFLD or NASH, optionally in conjunction with one or more dopamine D2 receptor agonists. The effective dosage will depend on the severity of the disease and the activity of the particular compound(s) employed, and is thus within the ordinary skill of the art to determine for any particular patient. However, the effective amount for treating NAFLD and NASH is generally between about 0.05 μg and about 5 mg/kg of body weight/day of D1 and/or D2 noradrenaline signal transduction inhibitors, and combinations thereof.

Optionally, circadian timed oral administration of gut anti-inflammatory agents (e.g., antibiotics, prebiotics, and/or probiotics) such as GPH agents described herein including gut-targeted antibiotics, ampicillin, indigestible carbohydrates, or probiotics in yogurts, tablets or the like for example, that reduce the pro-inflammatory gut microbiota in a dose range of 0.01 to 100 mg/kg body weight per day for a period sufficient to observe reversal of disease state progression (between about 2 and about 60 months) and/or administration of anti-diabetes agents pioglitazone, SGLT2 inhibitors, or GLP-1 receptor agonists (analogs) for a period sufficient to observe attenuation or reversal of disease state (between about 2 and about 60 months) at doses effective to treat type 2 diabetes but suboptimal to treat NAFLD or NASH (e.g., pioglitazone at 15 mg/day or less) may be co-administered with the dopamine neuronal activity enhancer. Furthermore, administration of agents that increase central dopaminergic neuronal activity plus pantethine with or without solubilized curcumin can be co-administered with anti-obesity agents, anti-hyperlipidemic agents (e.g., fibrates, FGF21) and/or anti-diabetes agents (e.g., pioglitazone, SGLT2 inhibitors, or GLP-1 receptor agonists (analogs)) for a period sufficient to attenuate or reverse NASH or NAFLD disease state (between about 2 and about 60 months). The GPH agent or anti-diabetes agent can be co-administered with the dopamine neuronal activity enhancer, administered sequentially, or can be administered as a component of the dopamine neuronal activity enhancer formulation. In one embodiment of the methods disclosed herein, a dopamine neuronal activity enhancer, probiotic and a prebiotic (such as solubilized curcumin and/or pantethine) are co-administered (either sequentially or simultaneously) to treat NASH or NAFLD.

In one embodiment, the agent(s) that increases central dopaminergic neuronal activity can be administered parenterally while the pantethine and curcumin and probiotics are administered orally preferably with the pantethine and curcumin in the same dosage form. In another embodiment, all agents are administered orally. In yet another embodiment the agent(s) that increases central dopaminergic activity is administered parenterally and the pantethine and curcumin are administered orally.

### Example 1: Treatment of NASH Mice with Bromocriptine Citrate Plus Pantethine with or Without Curcumin-Study 1

Male C57BL mice were fed a high fat high fructose diet for 19 weeks and then divided into 6 treatment groups as follows: (1) bromocriptine citrate (10 mg/kg); (2) bromocriptine plus solubilized curcumin (prepared as 700 mg of curcumin are added to a mixture containing 4 ml of phosphatidylcholine (of not less than 53% of total solution volume), between 3-6% of total volume of ethanol, up to 6% of total volume of lysophosphatidylcholine, and a mixture of caprylic/capric triglycerides, glyceryl stearate, oleic acid, ascorbyl palmitate, and DL-alpha-tocopherol each in amount of between 0.1 to about 40% (preferably between about 1 and about 15%) of total volume. The curcumin-phosphatidylcholine mixture is stirred until clear while being heated to between 50° C. to 60C. Optionally, add up to 10 ml of water and stir until solution is homogeneous)) (100 mg/kg). (3) bromocriptine plus pantethine (1100 mg/kg), (4) pantethine plus curcumin; (5) bromocriptine plus solubilized curcumin plus pantethine with each group administered the treatments within 4 hours of the onset of waking from the daily sleep cycle; and (6) such administered vehicle control and maintained on the test diet for an additional 6 weeks. After 6 weeks on the test diet and treatment, animals were sacrificed for analyses of body weight, body fat, blood glucose, and liver physiology. Relative to the vehicle control, the group receiving the bromocriptine plus solubilized curcumin plus pantethine (group (5)), exhibited the greatest reduction in liver weight (from 2.9 g in the vehicle control to 2.2 g in the bromocriptine plus solubilized curcumin plus pantethine group) (indirect measure of liver inflammation and fat reduction). The liver weight reduction of the bromocriptine plus solubilized curcumin plus pantethine group (at 24% reduction) was also greater than that of either the bromocriptine plus pantethine, bromocriptine plus solubilized curcumin, or pantethine plus solubilized curcumin groups (each at about 14% reduction). The treatment combination of bromocriptine plus solubilized curcumin plus pantethine produced the greatest reduction in a NASH propensity scale score (numeric value of the multiplicative of liver weight (g)×body weight (g)×body fat (mg)×liver triglyceride content (mg)×blood glucose level (mg/dl) with the vehicle value assigned 1.0 (maximum NASH propensity score)) relative to all other treatment groups: vehicle: 1.0, bromocriptine: 0.46, bromocriptine plus pantethine: 0.14, pantethine plus solubilized curcumin: 0.26, bromocriptine plus pantethine plus solubilized curcumin: 0.12.

Moreover, the NASH activity score (NAS) which is a measure of liver lobular inflammation, hepatocyte ballooning, and steatosis combined was reduced by 48% in the bromocriptine plus pantethine plus curcumin group relative to vehicle control animals. Finally, histological examination of the liver of the bromocriptine plus pantethine plus solubilized curcumin group demonstrated significant reduction in liver fibrosis relative to the vehicle control group. Also, addition of pantethine to bromocriptine treatment improved the NASH propensity scale score of bromocriptine treated animals from 0.46 to 0.14, a further 70% reduction versus bromocriptine alone. Both the bromocriptine plus pantethine and bromocriptine plus pantethine plus solubilized curcumin groups also reduced body fat (obesity) and fasting plasma glucose levels by approximately 43% and 33%, respectively, relative to vehicle controls. The bromocriptine plus pantethine and bromocriptine plus pantethine plus solubilized curcumin groups also improved glucose intolerance (total area under 2-hr glucose challenge (1.5 g/kg) curve) by approximately 50%.

The effect of bromocriptine plus pantethine on NASH and NASH related metabolic disorders is superior to either treatment alone and the effect of bromocriptine plus pantethine plus curcumin on NASH and NASH related metabolic disorders is superior to that of bromocriptine and pantethine alone.

### Example 2: Treatment of NASH Mice with Bromocriptine Citrate Plus Pantethine with or without Curcumin-Study 2

Livers isolated from the animals in Example 1 above were assayed for gene mRNA expression levels of proteins known to participate in the development and progression of NASH (via potentiation of inflammation and fibrosis) including Galectin 3, CCL2, CCR2, NOS2, and aSMA. The bromocriptine plus pantetheine plus solubilized curcumin treatment reduced the gene mRNA expression levels of Galectin 3, CCL2, CCR2, NOS2, and aSMA genes by 56%, 62%, 65%, 53%, and 63%, respectively, relative to the vehicle control group.

### Example 3: Administration of L-DOPA Plus Pantethine with or without Curcumin Formulation to Treat NASH-Study 1

Male C57BL mice were fed a high fat high fructose diet for 66 days and then divided into 4 treatment groups as follows: (1) pantethine (1100 mg/kg); (2) L-DOPA (20 mg/kg) plus pantethine; (3) L-DOPA plus solubilized curcumin (100 mg/kg) plus pantethine; and (4) vehicle control, and maintained on the test diet for an additional 32 days. After 32 days on the test diet, animals were sacrificed for analyses of body weight, body fat, blood glucose, and liver triglyceride. The group receiving L-DOPA with solubilized curcumin and pantethine exhibited the greatest reduction in a NASH propensity score (numeric score of liver triglyceride level×body weight×body fat×blood glucose level with vehicle set to 1.0) as follows: pantethine: 0.32; L-Dopa plus pantethine: 0.25; L-DOPA plus pantethine plus solubilized curcumin: 0.20. The treatment combination of L-DOPA plus pantethine produced a substantial reduction in NASH propensity scale score relative to vehicle controls of 75% while L-DOPA plus solubilized curcumin plus pantethine produced the greatest reductions in NASH propensity scale score of 80% (25% greater than L-DOPA plus pantethine (0.20 vs. 0.25)). The L-DOPA plus pantethine and L-DOPA plus pantethine plus solubilized curcumin groups also had reduced retroperitoneal body fat (obesity) by 29% and 41%, respectively, and reduced hyperglycemia by 21% and 28%, respectively relative to vehicle controls.

### Example 4: Administration of L-DOPA Plus Pantethine with or without Curcumin Formulation to Treat NASH-Study 2

Mice were fed a high fat high fructose diet for 12 weeks and then divided into 8 treatment groups as follows: (1) L-DOPA (20 mg/kg); (2) solubilized curcumin (100 mg/kg); (3) pantethine (1100 mg/kg); (4) L-DOPA plus pantethine; (5) L-DOPA plus solubilized curcumin, (6) pantethine plus curcumin; (7) L-DOPA plus solubilized curcumin plus pantethine; and (8) vehicle control, and maintained on the test diet for an additional 3 weeks. After 3 weeks on the test diet, animals were sacrificed for analyses of body weight, body fat, blood glucose, and liver physiology. Relative to the vehicle control group, the L-DOPA plus solubilized curcumin plus pantethine group (Group 7), exhibited the greatest reduction in liver weight (from 2.96 g in the vehicle control to 1.95 g in the L-DOPA plus solubilized curcumin plus pantethine group, a 34% reduction) (indirect measure of liver inflammation and fat reduction). Relative to the vehicle control group, the L-DOPA plus solubilized curcumin plus pantethine group exhibited the greatest reduction in the NASH propensity score (numeric value of the multiplicative of liver weight (g)×body weight (g)×body fat (mg)×blood glucose level (mg/dl) with the vehicle value assigned 1.0 (maximum NASH propensity score)) as follows: L-DOPA: 0.61; pantethine: 0.18; curcumin: 0.73; L-DOPA plus pantethine: 0.11; L-DOPA plus curcumin: 0.62; pantethine plus curcumin: 0.13; L-DOPA plus pantethine plus curcumin: 0.09.

The L-DOPA plus pantethine and L-DOPA plus pantethine plus solubilized curcumin treatment also reduced epididymal body fat (obesity) by 29% and 37%, respectively and hyperglycemia by 31% and 37%, respectively, relative to vehicle controls.

Analyses of gene mRNA expression levels in liver gene markers of fibrosis and inflammation are measured and determined to be substantially reduced in the L-DOPA plus pantetheine and more so in L-DOPA plus pantethine plus curcumin groups relative to vehicle controls. The liver reductions in these test parameters of the L-DOPA plus solubilized curcumin plus pantethine group is also greater than that of either the L-DOPA plus pantethine, L-DOPA plus solubilized curcumin, or pantethine plus solubilized curcumin groups. The treatment combination of bromocriptine plus solubilized curcumin plus pantethine plus probiotic produces the greatest reductions in measures of liver NASH (liver weight, liver fat content, and liver gene expression of inflammatory and fibrosis genes) body weight, body fat and blood glucose relative to all other treatment groups.

The effect of L-DOPA plus pantethine on NASH and NASH related metabolic disorders is superior to either treatment alone and the effect of L-DOPA plus pantethine plus curcumin on NASH and NASH related metabolic disorders is superior to that of L-DOPA and pantethine alone.

### Example 5. Administration of L-DOPA Plus Pantethine with or without Curcumin Formulation to Treat NASH Plus Metabolic Disorders

Mice are fed a high fat, high fructose diet for 16 weeks and then divided into 8 treatment groups as follows: (1) L-DOPA (20 mg/kg); (2) solubilized curcumin (100 mg/kg); (3) pantethine (1100 mg/kg); (4) L-DOPA plus pantethine; (5) L-DOPA plus solubilized curcumin; (6) pantethine plus curcumin; (7) L-DOPA plus solubilized curcumin plus pantethine; and (8) vehicle control, and maintained on the test diet for an additional 3 weeks. After 4 weeks on the test diet, animals are sacrificed for analyses of body weight, body fat, blood glucose, and liver physiology. Relative to vehicle control group the L-DOPA plus pantethine plus curcumin group exhibits the greatest reduction in NASH, type 2 diabetes, obesity, and indices of cardiovascular disease (vascular inflammation). The L-DOPA plus pantethine group also improves these conditions relative to vehicle control but not to the extent of the L-DOPA plus pantethine plus curcumin group.

### Example 6. Dopaminergic Neuronal Activity Enhancer Formulations to Treat NASH

A pharmaceutical package for treating NASH or NAFLD includes an agent that increases central dopaminergic neuronal activity such as bromocriptine of between 0.1 to 5 mg formulated with excipients into a sublingual dosage form and an oral dosage form containing pantethine between 100 to 1500 mg and an oral dosage form of solubilized curcumin (either liquid or subsequently made into a powder form) between 10 to 1000 mg. The pantethine and solubilized curcumin doses can be made into a single dosage form of either liquid or solid form containing both agents. Additionally, an oral dosage form containing probiotic can be included with the pharmaceutical package either alone or combined with the pantethine or curcumin dosage form or pantethine plus curcumin dosage form. The formulations are effective in treating NAFLD and NASH including metabolic disorders.

### Example 7. Dopaminergic Neuronal Activity Enhancer Formulations to Treat NASH

A pharmaceutical formulation for treating NASH or NAFLD includes a dosage form of L-DOPA between 20 to 2000 mg (e.g., within Mucuna pruriens extract powder at 120 to 7000 mg), pantethine in an amount of between 100 to 1500 mg in powder form with a binder such as microcrystalline cellulose, and solubilized curcumin made into powder form in an amount of between 20 to 2000 mg. A pharmaceutical formulation for treating NASH or NAFLD includes a dosage form of a biological source containing L-DOPA in an amount of between 20 to 2000 mg in powder form, pantethine between 100 to 1500 mg in powder form with a binder such as microcrystalline cellulose, and solubilized curcumin made into powder form between 20 to 2000 mg. The formulation is prepared as a tablet.

### Exmple 8. Dopaminergic Neuronal Activity Enhancer Formulations to Treat NASH

A pharmaceutical package for treating NASH or NAFLD includes (1) a dosage form of L-DOPA between 20 to 2000 mg (e.g., within Mucuna pruriens extract powder at 120 to 7000 mg); (2) a dosage form of pantethine between 100 to 1500 mg in either liquid (water) or powder form with binder such as microcrystalline cellulose; and (3) a dosage form of solubilized curcumin either as liquid or made into powder form between 20 to 2000 mg. A pharmaceutical package for treating NASH or NAFLD includes (1) a dosage form of a biological source that contains L-DOPA between 20 to 2000 mg (e.g., within Mucuna pruriens extract powder at 120 to 7000 mg); (2) a dosage form of pantethine between 100 to 1500 mg in either liquid (water) or powder form with binder such as microcrystalline cellulose; and (3) a dosage form of solubilized curcumin either as liquid or made into powder form between 20 to 2000 mg.

### Example 9. Resetting the Circadian Rhythm of Central Dopaminergic Neuronal Activity with L-DOPA Plus Pantethine Plus Curcumin

Animals with a diminished circadian peak of central dopaminergic activity are treated within 4 hours of waking from the daily sleep cycle with L-DOPA, pantethine, curcumin, L-DOPA plus pantethine, L-DOPA plus curcumin, pantethine plus curcumin, L-DOPA plus pantethine plus curcumin or vehicle control. Relative to vehicle control, the L-DOPA plus pantethine plus curcumin group exhibits the best restoration/reinstatement of the circadian peak in the central dopaminergic neuronal activity rhythm (i.e., closest to the circadian peak in central dopaminergic neuronal activity of a healthy animal).

The results achieved with the present method of treatment indicate that the combination of appropriately timed agents that increase dopaminergic neuronal activity administered with pantethine and solubilized curcumin has the unique ability to effectively treat NASH and NAFLD and to provide a level of improvement not achievable with any of these therapies alone.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-36. (canceled)

37. A method of treating non-alcoholic steatohepatitis (NASH) in a subject in need thereof, which comprises administering to a subject in need of such treatment a biological source of L-DOPA,-pantethine, and curcumin, said co-administration taking place between 0400 and 1200 hours.

38. The method of claim 37, wherein the subject is also afflicted with a metabolic disorder selected from the group consisting of obesity, prediabetes, glucose intolerance, hypertension, dyslipidemia, type 2 diabetes, cardiovascular disease associated with NASH, comprising administering to a subject in need of such treatment Mucuna pruriens containing L-DOPA, pantethine, and optionally solubilized curcumin, said co-administration taking place within 4 hours of waking in the morning wherein the formulation treats the metabolic disorder and the NASH.

39. The method of claim 37, wherein the biological source of L-DOPA is broad bean or Mucuna pruriens.

40. The method of claim 37, wherein the biological source of L-DOPA is Mucuna pruriens delivered at between 6 and 150 mg/kg.

41. The method of claim 38, wherein the curcumin is solubilized curcumin.

42. The method of claim 41, wherein the solubilized curcumin comprises a mixture of phosphatidylcholine, caprylic/capric triglyceride, alcohol, glyceryl stearate, oleic acid, ascorbyl palmitate, and tocopherol.

43. The method of claim 42, which comprises administering the curcumin to the subject in an amount of between about 0.1 mg/kg to about 0.07 g/kg of body weight per day.

44. The method of claim 42, which further comprises administering a probiotic to the subject.

45. The method of claim 38, wherein the formulation delivers a dosage of between 1 and 20 mg/kg body weight of L-DOPA, between 5 and 100 mg/kg body weight pantethine, and between 2 and 30 mg/kg body weight curcumin.

46. The method of claim 38, wherein the formulation comprises at least one solid dosage form.

47. The method of claim 46, wherein the solid dosage form is selected from the group consisting of a tablet, a capsule or a powder.

48. A pharmaceutical dosage comprising a biological source of L-DOPA, pantethine and curcumin.

49. The pharmaceutical dosage of claim 48, wherein the dosage is in the form of a tablet, a capsule or a powder.

50. The pharmaceutical dosage of claim 49, wherein the curcumin is solubilized and includes a mixture of phosphatidylcholine, caprylic/capric triglyceride, alcohol, glyceryl stearate, oleic acid, ascorbyl palmitate, and tocopherol.

51. The pharmaceutical dosage of claim 48, comprising between 20 and 2000 mg L-DOPA, between 100 and 1500 mg pantethine, between 2 and 2,000 mg curcumin and a binder.

52. The pharmaceutical dosage of claim 48, wherein the biological source of L-DOPA is Mucuna Pruriens extract.

53. The pharmaceutical dosage of claim 52, comprising Mucuna pruriens extract in an amount between 120 and 5000 mg., pantethine in an amount between 100 and 1500 m, solubilized curcumin in an amount between 2 and 2000 mg; and a binder.

54. A method of treating metabolic disorders in a subject in need thereof which comprises co-administering to a subject in need of such treatment in a single formulation or separately (i) Mucuna pruriens containing L-DOPA, (ii) pantethine, and (ii) curcumin, said co-administration taking place within 4 hours of waking in the morning.

55. The method of claim 54, wherein the metabolic disorder is pre-diabetes.

56. The method of claim 54, wherein the metabolic disorder is hypertension.
